(19) United States
(12) Patent Application Publication
(10) Pub. No.: US 2026/0253275 A1
(43) Pub. Date: Aug. 27, 2026

(54) INFORMATION PROCESSING DEVICE, IMAGE GENERATION METHOD, AND NON-TRANSITORY COMPUTER-READABLE MEDIUM

(71) Applicants: NEC Corporation, Tokyo (JP); NEC Solution Innovators, Ltd., Tokyo (JP)

(72) Inventors: Keisuke SUZUKI, Tokyo (JP); Mitsuru NOMA, Tokyo (JP); Osamu HISAMATSU, Tokyo (JP); Yoshinori WADA, Tokyo (JP); Masayuki ITABASHI, Tokyo (JP)

(73) Assignees: NEC Corporation, Tokyo (JP); NEC Solution Innovators, Ltd., Tokyo (JP)

(21) Appl. No.: 19/537,795

(22) Filed: Feb. 12, 2026

(30) Foreign Application Priority Data
Feb. 25, 2025 (JP) 2025-028234

(51) Int. Cl. G06T 11/26; A61B 5/339; G06T 3/40; G16H 30/40

(52) U.S. Cl. CPC G06T 11/26; A61B 5/339; G06T 3/40; G16H 30/40; A61B 2560/0238; G06T 2210/32; G06T 2210/41

(57) ABSTRACT

An information processing device includes a digitizing unit for generating numerical data indicating a time-series change of a biological signal from first image data indicating a waveform of the biological signal, and an image generation unit for generating second image data indicating a waveform of the biological signal by using the generated numerical data. The generated second image data can be used as training data for machine learning or can be used for decision making by a medical worker in diagnosis of a subject to be measured for the biological signal.

### INCORPORATION BY REFERENCE

This application is based upon and claims the benefit of priority from Japanese patent application No. 2025-028234, filed on Feb. 25, 2025, the disclosure of which is incorporated herein in its entirety by reference.

### TECHNICAL FIELD

The present disclosure relates to an information processing device, an image generation method, and an image generation program.

### BACKGROUND ART

Various biological signal data are utilized in various applications.

For example, JP 2020-130772 A below discloses a biological signal processing device that generates data for automatic analysis capable of efficiently performing deep learning of a relationship between a plurality of types of leads in an electrocardiogram. More specifically, in the biological signal processing device of JP 2020-130772 A, electrocardiogram data output from an electrocardiograph acquired, and image data representing waveform information regarding a plurality of types of leads is generated from the acquired electrocardiogram data. The image data generated in this manner is used for deep learning as the above-described data for automatic analysis.

### SUMMARY

Here, waveforms of various biological signals including the electrocardiogram are often stored as image data in the first place. The waveform of the biological signal may be printed on a recording sheet and stored as an image. However, in the biological signal processing device of JP 2020-130772 A, the electrocardiogram data output from the electrocardiograph is necessary for generating the data for automatic analysis, and the data for automatic analysis cannot be generated from the image data of the electrocardiogram or the image on which the electrocardiogram is printed.

The image data indicating the waveform of the biological signal can be acquired by a method of using a general biological signal output device or reading a recording sheet on which the waveform of the biological signal is printed with a scanner without using the biological signal processing device of JP 2020-130772 A. However, the format and display mode of the image data indicating the waveform of the biological signal are often different depending on a vendor of the measurement device and/or the output device of the biological signal. Then, in a series of deep learning, if image data having different formats and display modes are used together, a problem such as deterioration in learning accuracy may occur.

As described above, the image data indicating the waveform of the biological signal is widely spread and easily available, but there is a problem in its availability. The present disclosure has been made in view of such a problem, and an example object of the present disclosure is to provide a technique capable of enhancing the availability of image data indicating a waveform of a biological signal.

An information processing device according to an example aspect of the present disclosure includes a digitizing means for generating numerical data indicating a time-series change of a biological signal from first image data indicating a waveform of the biological signal, and an image generation means for generating second image data indicating a waveform of the biological signal by using the numerical data.

In an image generation method according to an example aspect of the present disclosure, at least one processor executes digitizing processing of generating numerical data indicating a time-series change of a biological signal from first image data indicating a waveform of the biological signal, and image generation processing of generating second image data indicating a waveform of the biological signal by using the numerical data.

An image generation program according to an example aspect of the present disclosure causes a computer to function as a digitizing means for generating numerical data indicating a time-series change of a biological signal from first image data indicating a waveform of the biological signal, and an image generation means for generating second image data indicating a waveform of the biological signal using the numerical data.

According to an example aspect of the present disclosure, there is an example effect that the availability of image data indicating a waveform of a biological signal can be enhanced.

### BRIEF DESCRIPTION OF THE DRAWINGS

FIG. 1 is a block diagram illustrating a configuration of an information processing device according to the present disclosure;

FIG. 2 is a flowchart illustrating a flow of an image generation method according to the present disclosure;

FIG. 3 is a block diagram illustrating a configuration of another information processing device according to the present disclosure;

FIG. 4 is a diagram illustrating an example of image data acquired by a data acquisition unit;

FIG. 5 is a diagram illustrating image data generated by performing binarization processing on a second area of the image data illustrated in FIG. 4;

FIG. 6 is a diagram illustrating an example of an electrocardiogram waveform to be digitized;

FIG. 7 is a diagram illustrating an example of a method of generating second image data;

FIG. 8 is a diagram illustrating another example of a method of generating the second image data;

FIG. 9 is a flowchart illustrating an example of processing performed by the information processing device illustrated in FIG. 3; and

FIG. 10 is a block diagram illustrating a configuration of a computer that functions as the information processing device according to the present disclosure.

### EXAMPLE EMBODIMENT

Hereinafter, example embodiments of the present invention will be described. However, the present invention is not limited to the following illustrative example embodiments, and various modifications can be made within a scope described in the claims. For example, example embodiments obtained by appropriately combining techniques (some or all of things or methods) adopted in the following illustrative example embodiments can also be included in the scope of the present invention. Example embodiments obtained by appropriately omitting some of the techniques adopted in the following illustrative example embodiments can also be included in the scope of the present invention. Effects mentioned in the following illustrative example embodiments are examples of effects expected in the illustrative example embodiments, and do not define extension of the present invention. That is, illustrative example embodiments that do not achieve the effects mentioned in each of the illustrative example embodiments described below can also be included in the scope of the present invention.

### First Illustrative Example Embodiment

A first illustrative example embodiment that is an example of the example embodiments of the present invention will be described in detail with reference to the drawings. The present illustrative example embodiment is a basic form of each illustrative example embodiment to be described below. An application range of each technique adopted in the present illustrative example embodiment is not limited to the present illustrative example embodiment. That is, each technique adopted in the present illustrative example embodiment can also be adopted in another illustrative example embodiment included in the present disclosure within a range in which no particular technical problem occurs. Each technology illustrated in the drawings referred to for describing the present illustrative example embodiment can also be adopted in other illustrative example embodiments included in the present disclosure within a range in which no particular technical problem occurs.

### (Configuration of Information Processing Device 1)

A configuration of an information processing device 1 according to the present illustrative example embodiment will be described with reference to FIG. 1. FIG. 1 is a block diagram illustrating the configuration of the information processing device 1. As illustrated in FIG. 1, the information processing device 1 includes a digitizing unit 101 and an image generation unit 102.

The digitizing unit 101 generates numerical data indicating a time-series change of the biological signal from first image data indicating the waveform of the biological signal. Here, the “biological signal” is a signal derived from a living body and reflecting a state or a function of the living body or an organ thereof. For example, signals such as an electrocardiogram, an electroencephalogram, a magnetoencephalogram, a heart rate, a body temperature, and a blood pressure can be used as the biological signal. Here, “numerical data” means data including numerical values as elements.

The time-series biological signal can be expressed as a waveform, and the “first image data” is data indicating such a waveform. The first image data may be any data that can generate numerical data from the image data. For example, the first image data may be image data in a format that allows conversion between numerical values and images, such as medical waveform form at encoding rules (MFER). However, the first image data is not necessarily image data in a special format such as the MFER format. For example, the first image data may be data in a format such as PNG (Portable Network Graphics), PDF (Portable Document Format), JPEG (Joint Photographic Experts Group), BMP (Bitmap), or GIF (Graphics Interchange Format). A method of generating numerical data from image data in such a format will be described in a second illustrative example embodiment. For example, the first image data may be generated by reading a recording sheet on which a waveform of the biological signal is printed with a scanner, may be output by a measurement device of the biological signal, or may be output by an output device connected to the measurement device of the biological signal.

The image generation unit 102 generates second image data indicating the waveform of the biological signal using the numerical data generated by the digitizing unit 101. Here, the “second image data” is data indicating a waveform of a time-series biological signal, similarly to the first image data described above. The first image data and the second image data both show the same waveform, but are different data. Since the second image data is generated using the numerical data generated by the digitizing unit 101, the image generation unit 102 can generate the second image data in a format different from that of the first image data, or can generate the second image data (for example, in a different layout) in a display mode different from that of the first image data. In the following description, the fact that at least one of the format and the display mode is different may be expressed as “the format and the like are different”.

As described above, the information processing device 1 according to the present illustrative example embodiment employs a configuration including the digitizing unit 101 that generates numerical data indicating a time-series change of a biological signal from first image data indicating a waveform of the biological signal, and the image generation unit 102 that generates second image data indicating a waveform of the biological signal using the numerical data generated by the digitizing unit 101 based on a detection result of the digitizing unit 101.

According to the above configuration, from the first image data, the second image data having the same waveform as that of the first image data but having a format or the like different from that of the first image data can be generated. Then, the second image data can be used for analysis of the biological signal, for machine learning, or for medical worker's decision making in diagnosis of the person to be measured for the biological signal, instead of or together with the first image data. Therefore, according to the above configuration, it is possible to increase the availability of the image data indicating the waveform of the biological signal.

### (Image Generation Program)

The functions of the information processing device 1 described above can also be achieved by a program. An image generation program according to the present illustrative example embodiment causes a computer to function as a digitizing means for generating numerical data indicating a time-series change of a biological signal from first image data indicating a waveform of the biological signal, and an image generation means for generating second image data indicating a waveform of the biological signal using the numerical data. According to this image generation program, it is possible to increase the availability of the image data indicating the waveform of the biological signal.

### (Flow of Image Generation Method)

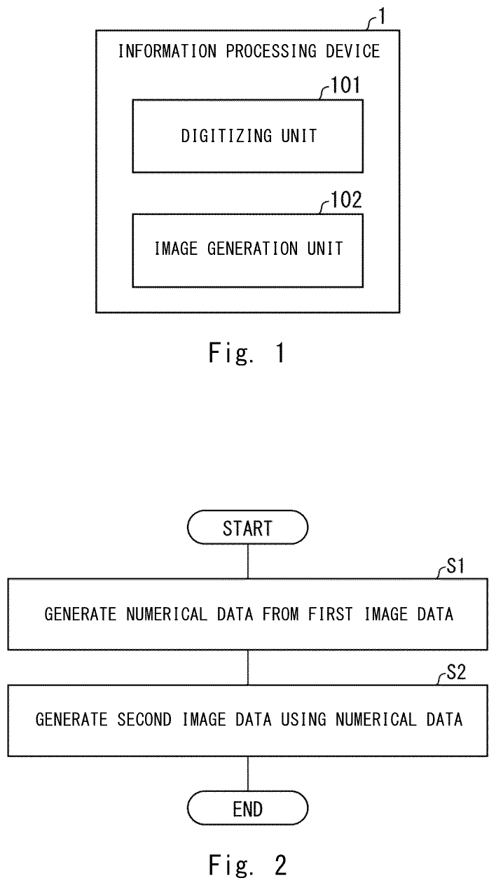

A flow of an image generation method according to the present illustrative example embodiment will be described with reference to FIG. 2. FIG. 2 is a flowchart illustrating the flow of the image generation method. An entity that executes steps in this image generation method may be a processor included in the information processing device 1, may be a processor included in another device, or may be processors located in different devices for each step of the process.

In S1 (digitizing processing), at least one processor generates numerical data indicating a time-series change of a biological signal from first image data indicating a waveform of the biological signal.

In S2 (image generation processing), at least one processor generates second image data indicating a waveform of the biological signal using the numerical data generated in S1.

As described above, in the image generation method according to the present illustrative example embodiment, a configuration is adopted in which at least one processor executes the digitizing processing of generating numerical data indicating the time-series change of the biological signal from the first image data indicating the waveform of the biological signal, and the image generation processing of generating the second image data indicating the waveform of the biological signal using the generated numerical data. According to this image generation method, it is possible to increase the availability of the image data indicating the waveform of the biological signal.

### Second Illustrative Example Embodiment

A second illustrative example embodiment that is an example of the example embodiments of the present invention will be described in detail with reference to the drawings. Components having the same functions as the components described in the above-described illustrative example embodiment are denoted by the same reference signs, and the description thereof will be appropriately omitted. An application range of each technique adopted in the present illustrative example embodiment is not limited to the present illustrative example embodiment. That is, each technique adopted in the present illustrative example embodiment can also be adopted in another illustrative example embodiment included in the present disclosure within a range in which no particular technical problem occurs. Each technique illustrated in each of the drawings referred to for describing the present illustrative example embodiment can be employed in the other illustrative example embodiments included in the present disclosure within the scope in which no particular technical problem occurs.

### (Configuration of Information Processing Device 1A)

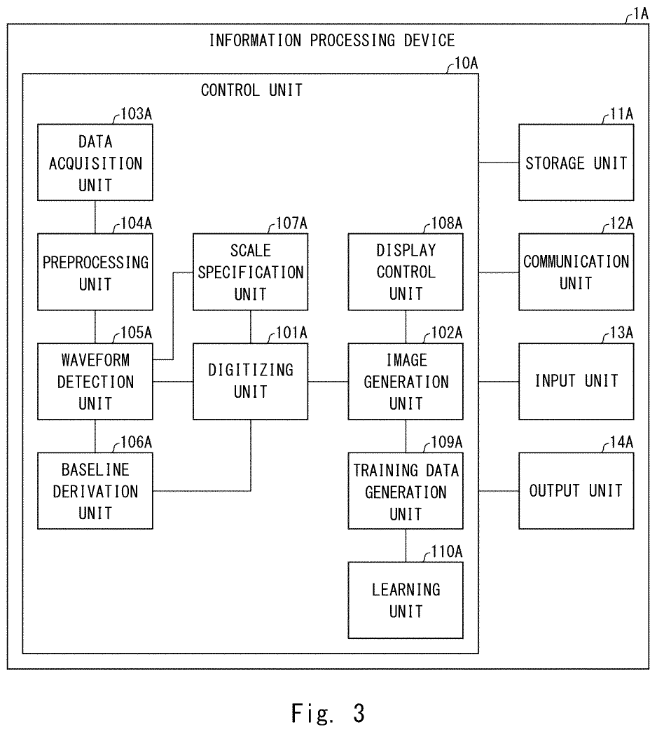

A configuration of an information processing device 1A according to the present illustrative example embodiment will be described with reference to FIG. 3. FIG. 3 is a block diagram illustrating a configuration of the information processing device 1A. The information processing device 1A is a device having a function of generating second image data of a biological signal from first image data of the biological signal.

As illustrated, the information processing device 1A includes a control unit 10A for integrally controlling each unit of the information processing device 1A, and a storage unit 11A for storing various types of data to be used by the information processing device 1A. The information processing device 1A includes a communication unit 12A for the information processing device 1A to communicate with another device, an input unit 13A for accepting an input to the information processing device 1A, and an output unit 14A for the information processing device 1A to output data. The control unit 10A includes a digitizing unit 101A, an image generation unit 102A, a data acquisition unit 103A, a preprocessing unit 104A, a waveform detection unit 105A, a baseline derivation unit 106A, a scale specification unit 107A, a display control unit 108A, a training data generation unit 109A, and a learning unit 110A.

The digitizing unit 101A generates numerical data indicating the time-series change of the biological signal from the first image data indicating the waveform of the biological signal, similarly to the digitizing unit 101 of the first illustrative example embodiment. Image data of any biological signal is used as the first image data. Hereinafter, an example in which the image data of the electrocardiogram is used as the first image data will be mainly described, but the “electrocardiogram” in the following description can be basically replaced with any “biological signal”. In the present illustrative example embodiment, an example in which image data in a general image format such as PNG or PDF is used as the first image data instead of image data in a special format such as a format in which conversion between numerical values and images is possible will be described.

Similarly to the image generation unit 102 of the first illustrative example embodiment, the image generation unit 102A generates the second image data indicating the waveform of the biological signal using the numerical data generated by the digitizing unit 101A. Details of the method of generating the second image data will be described later.

The data acquisition unit 103A acquires image data (hereinafter, referred to as original image data) that is a source of the first image data. Although details will be described below, the preprocessing unit 104A performs preprocessing of removing elements other than the waveform on the original image data acquired by the data acquisition unit 103A, thereby generating the first image data. The data acquisition unit 103A may acquire the first image data instead of acquiring the original image data. In this case, the preprocessing unit 104A is omitted.

The preprocessing unit 104A performs preprocessing of removing elements other than the waveform on the original image data acquired by the data acquisition unit 103A to generate the first image data. Details of the preprocessing executed by the preprocessing unit 104A will be described later.

The waveform detection unit 105A detects a pixel group relevant to the waveform portion of the biological signal from the first image data. In the image data indicating the waveform, regardless of its format or the like, a portion of the waveform and a portion of its background can be identified. Therefore, even if the first image data is not image data of a special format such as MFER, the waveform detection unit 105A can detect a pixel group relevant to the portion of the waveform from the first image data.

The baseline derivation unit 106A derives a baseline passing through a plurality of points at which the biological signal has a predetermined reference value in the waveform of the biological signal. For example, in a case where the biological signal is an electrocardiogram, the baseline derivation unit 106A may detect a portion where the cardiac potential becomes linear in the waveform of the electrocardiogram, and derive the baseline based on the potential of the linear portion. For example, the baseline derivation unit 106A may detect a plurality of points at which the cardiac potential becomes 0 in the waveform of the electrocardiogram, and derive a straight line passing through each detected point as a baseline.

The scale specification unit 107A specifies a scale in the first image data. The digitizing unit 101A generates numerical data using the specified scale. A method of specifying a scale and a method of generating numerical data using the specified scale will be described later.

The display control unit 108A causes the display device to display the second image data generated by the image generation unit 102A. The display device may be included in the information processing device 1A or may be a device outside the information processing device 1A.

The training data generation unit 109A generates training data for machine learning of the inference model using the second image data generated by the image generation unit 102A. This inference model is a model for inferring the state of the subject based on image data indicating the waveform of the biological signal of the subject. Specifically, the training data generation unit 109A generates training data by associating a correct inference result (which can also be referred to as a correct answer label) with the generated second image data. Any algorithm of the inference model may be used. For example, the training data generation unit 109A may generate training data for machine learning of an inference model such as a deep neural network. Any inference content of the inference model may be also used. For example, the inference model may be a model for inferring the presence or absence of a predetermined disease.

In a case where the training data of the inference model for inferring the presence or absence of the predetermined disease is generated, the training data generation unit 109A sets the second image data derived from the biological signal of the person having the predetermined disease as the training data in association with a correct answer label indicating that the person has the predetermined disease. On the other hand, the training data generation unit 109A sets the second image data derived from the biological signal of the person who does not have the predetermined disease as the training data in association with the correct answer label indicating that the person does not have the predetermined disease.

The learning unit 110A generates the inference model by performing machine learning using the training data generated by the training data generation unit 109A. Specifically, the learning unit 110A repeats processing of updating the parameter of the inference model so as to reduce a difference between an inference result obtained by inputting the second image data included in the training data to the inference model and the correct answer label associated with the training data for a plurality of training data. As a result, by inputting the image data indicating the waveform of the biological signal of the subject, the inference model that outputs the inference result regarding the state of the subject is generated.

The information processing device 1A may include an inference unit (inference means) that performs inference using a machine-learned inference model by the learning unit 110A. In this case, the image data input to the information processing device 1A for inference only needs to indicate the waveform of the biological signal of the subject and include information necessary for inference, and any format and the like are used as long as the digitizing unit 101A can generate numerical data. This is because the digitizing unit 101A can generate numerical data from the first image data as long as the first image data is relevant to a layout candidate assumed in advance. Then, this is because the image generation unit 102A can generate second image data in a format that can be used as input data of the inference model from the generated numerical data.

As described above, also in the information processing device 1A according to the present illustrative example embodiment, similarly to the information processing device 1, a configuration is adopted in which the information processing device 1A includes the digitizing unit 101A that generates numerical data indicating a time-series change of a biological signal from first image data indicating a waveform of the biological signal, and the image generation unit 102 that generates second image data indicating a waveform of the biological signal using the numerical data generated by the digitizing unit 101A. Therefore, also in the information processing device 1A, it is possible to increase the availability of the image data indicating the waveform of the biological signal.

### Preprocessing Example 1: Extraction of Part of Original Image Data

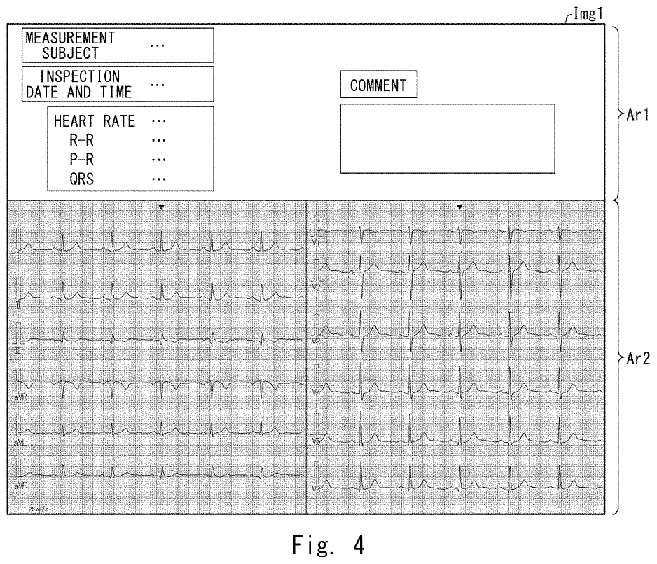

An example of preprocessing by the preprocessing unit 104A will be described with reference to FIG. 4. FIG. 4 is a diagram illustrating an example of image data (that is, the above-described original image data) acquired by the data acquisition unit 103A. Original image data Img1 illustrated in FIG. 4 is image data of an electrocardiogram. More specifically, the original image data Img1 is image data of a 12-lead electrocardiogram, and includes 12 waveforms relevant to 12 types of leads.

As illustrated in the drawing, the image area of the original image data Img1 is divided into a first area Ar1 in which electrocardiogram related information such as the name of the subject to be measured in the electrocardiogram, the measurement date and time, the analysis result, and the comment of the laboratory technician is displayed, and a second area Ar2 in which the electrocardiogram waveforms of 12 leads are displayed.

In a case where such original image data Img1 is acquired, the preprocessing unit 104A may extract the second area Ar2 from the original image data Img1. For example, in a case where the ranges occupied by the first and second areas are determined in the original image data Img1, the preprocessing unit 104A may extract the range of the second area Ar2 from the original image data Img1. For example, the preprocessing unit 104A may analyze the original image data Img1 to specify the range of the second area Ar2, and extract the specified range from the original image data Img1. The extraction range may be designated by the user. In this case, the preprocessing unit 104A may extract the range designated by the user from the original image data Img1 via the communication unit 12A or the input unit 13A.

As described above, the preprocessing unit 104A may perform processing of extracting an area where a waveform is drawn in the original image data as preprocessing of removing elements other than the waveform from the original image data. As a result, it is possible to eliminate the influence of the area where the waveform is not drawn in the original image data and to facilitate or improve the accuracy of the waveform detection by the waveform detection unit 105A.

### Preprocessing Example 2: Removal of Grid Line of Background of Waveform

Here, in the original image data Img1 illustrated in FIG. 4, a lattice-like grid line indicating the scale of the waveform is drawn on the background of the electrocardiogram waveform. Such grid lines can also interfere with the detection of the waveform by the waveform detection unit 105A. Therefore, the preprocessing unit 104A may perform preprocessing of removing the background grid line of the electrocardiogram waveform.

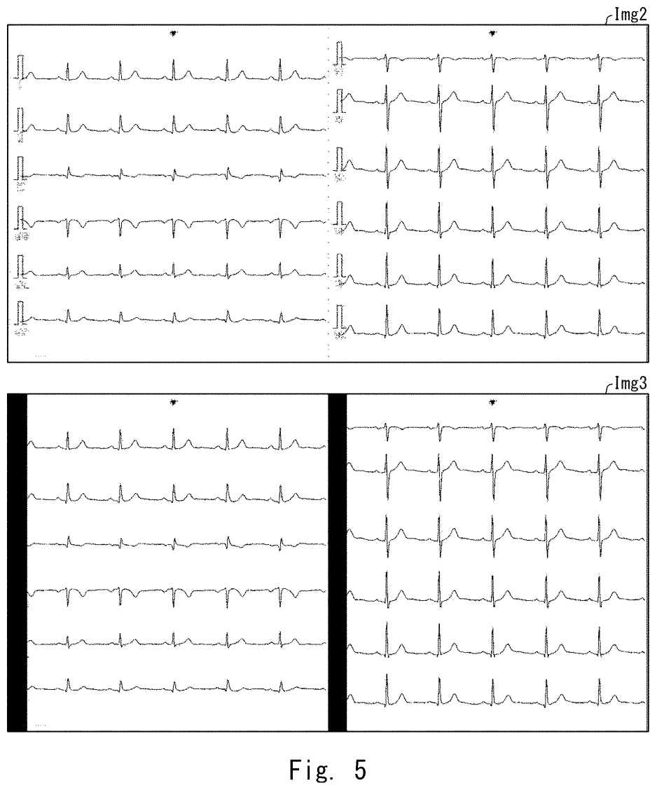

For example, the preprocessing unit 104A may remove the grid line of the background of the electrocardiogram waveform by binarizing the original image data. FIG. 5 is a diagram illustrating image data Img2 generated by performing the binarization processing on the second area Ar2 in the original image data Img1 illustrated in FIG. 4. By appropriately setting the binarization threshold, the image data Img2 from which the grid line drawn on the background of the electrocardiogram waveform is removed is obtained.

The image data Img2 includes, in addition to the electrocardiogram waveforms of 12 leads, symbols such as “I” and “II” indicating the type of each lead, and waveforms called rectangular waves or calibration waves. The preprocessing unit 104A may remove such an area where information other than the waveform is displayed.

For example, the preprocessing unit 104A may perform mask processing on an area where information other than the waveform is displayed. Image data Img3 illustrated in FIG. 5 is an example of image data generated by performing mask processing on an area where information other than a waveform is displayed in the image data Img2. Since what area the information other than the waveform is displayed is usually determined, the preprocessing unit 104A may set the area where such information is displayed as a target of the mask processing.

The preprocessing unit 104A may detect characters and the like other than the waveform by processing such as optical character recognition (OCR) and remove the detected characters and the like. The preprocessing unit 104A may detect and remove a figure other than the waveform by pattern matching or the like. In a case where the display color is different between the waveform and the portion other than the waveform, the preprocessing unit 104A may detect and remove the portion other than the waveform based on the color.

In the image data Img3 of FIG. 5, the area subjected to the mask processing is illustrated in black, but the mask processing may be processing of setting each pixel included in the target area to white (pixel value of zero). Instead of performing the mask processing, the area to be detected by the waveform detection unit 105A may be narrowed down to an area where information such as symbols other than the waveform is not displayed. In a case where a plurality of waveforms are included in the original image data, the preprocessing unit 104A may perform preprocessing of cutting out each area where each waveform appears.

As described above, the information processing device 1A includes the preprocessing unit 104A that generates first image data by performing preprocessing of removing elements other than a waveform on original image data that is a source of the first image data. As a result, in addition to the effect obtained by the information processing device 1, it is possible to eliminate the influence of elements other than the waveform in the original image data and to facilitate or improve the accuracy of the detection of the waveform by the waveform detection unit 105A.

### (Detection of Pixel Group Relevant to Waveform Portion)

The waveform detection unit 105A detects a pixel group relevant to a portion of the waveform of the electrocardiogram from the image data generated by the preprocessing unit 104A as described above. In the detection of the pixel group, the waveform detection unit 105A assigns a coordinate value to each pixel included in the image data. For example, the waveform detection unit 105A may assign a coordinate value to each pixel of the image data, assuming that the coordinates of the pixel at the reference position such as the upper left end of the image data are (0,0), the right direction of the image data is the positive direction of the x axis, the downward direction is the positive direction of the y axis, and the interval between adjacent pixels is 1. As a result, the pixels included in the image data can be represented by coordinate values relevant to the positions, and the pixels relevant to the portions of the waveform can be detected using the coordinate values.

A method of detecting the pixel relevant to the portion of the waveform is not particularly limited. For example, the waveform detection unit 105A may detect a pixel group including adjacent pixels in the image data as a pixel group relevant to a portion of the waveform of the electrocardiogram. As in the image data Img3 of FIG. 5, in a case where the target is the image data generated by the preprocessing that excludes all the elements other than the waveform, it is possible to detect the pixel group relevant to the portion of the waveform of the electrocardiogram by such simple processing. From the image data Img3, 12 pixel groups relevant to the 12 types of lead are detected.

The waveform detection unit 105A can also detect a pixel group relevant to a portion of a waveform from image data including elements other than the waveform, like the image data Img2 of FIG. 5. In this case, the waveform detection unit 105A may detect, as the pixel group relevant to the waveform portion of the electrocardiogram, a pixel group that satisfies a condition specific to the pixel group relevant to the waveform portion among the pixel groups including the adjacent pixels in the image data. For example, the waveform detection unit 105A may set a pixel group having the number of elements equal to or larger than a threshold as a pixel group relevant to a portion of the waveform of the electrocardiogram. For example, the waveform detection unit 105A may set a pixel group in which the distance from the left end pixel to the right end pixel is equal to or more than a threshold as a pixel group relevant to a portion of the waveform of the electrocardiogram. By these processes, a pixel group relevant to an element such as a character or a symbol can be excluded.

Here, in the image data Img2 of FIG. 5, a rectangular waveform is drawn at the left end of the twelve electrocardiogram waveforms. This waveform is called a rectangular wave or a calibration wave as described above, and is an object for calibration. In the image data Img2, since the electrocardiogram waveform intersects the calibration wave, in a case where a pixel group is detected from the image data Img2 under the condition of “a pixel group including adjacent pixels”, a pixel group including pixels including the electrocardiogram waveform and pixels including the calibration wave is detected.

Therefore, the waveform detection unit 105A may distinguish and detect the electrocardiogram waveform and the calibration wave. Since the shapes of the electrocardiogram waveform and the calibration wave are clearly different from each other, it is possible to distinguish and detect the electrocardiogram waveform and the calibration wave based on the difference in shape. For example, the waveform detection unit 105A may detect a pixel group including a calibration wave from image data by pattern matching or the like. In this case, the waveform detection unit 105A removes each pixel of the pixel group including the calibration wave from the pixel group including the pixels including the electrocardiogram waveform and the pixels including the calibration wave, and detects the rest pixels as the pixel group relevant to the waveform portion of the electrocardiogram.

It is not always necessary to cause the waveform detection unit 105A to detect the pixel group including the calibration wave. A detection unit that detects a pixel group including the calibration wave may be provided separately from the waveform detection unit 105A.

### (Method of Generating Numerical Data)

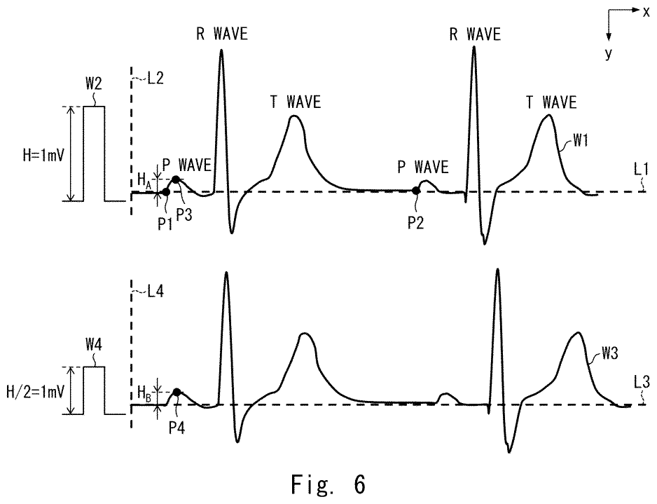

A method of generating numerical data by the digitizing unit 101A will be described with reference to FIG. 6. FIG. 6 is a diagram illustrating an example of an electrocardiogram waveform to be digitized. In a waveform W1 illustrated in FIG. 6, waveforms called a P wave, an R wave, and a T wave are periodically repeated. A calibration wave W2 is drawn in association with the waveform W1. As illustrated in the drawing, the calibration wave W2 is drawn in such a size that the height H becomes 1 m V.

In generating the numerical data, in a case where the baseline derivation unit 106A derives the baseline, the scale specification unit 107A specifies the scale. Hereinafter, a method of deriving the baseline and a method of specifying the scale will be sequentially described.

### (Method of Deriving Baseline)

The baseline in the electrocardiogram is a linear portion in the electrocardiogram. The baseline in the electrocardiogram represents a state in which there is no electrical activity in the myocardium. That is, the value of the potential on the baseline in the electrocardiogram is zero. For example, the baseline derivation unit 106A may detect each pixel located at the starting point of the P wave among the pixels included in the pixel group detected by the waveform detection unit 105A, calculate an average value μ of the y coordinates of these pixels, and derive a straight line represented by an expression of y=μ as a baseline. The pixel located at the starting point of the P wave can be detected by analyzing the coordinate value of each pixel included in the pixel group detected by the waveform detection unit 105A.

For example, the baseline derivation unit 106A may detect a pixel located at the start point of the P wave and a pixel located at the start point of the next P wave, and derive a straight line passing through these points as a baseline. In this case, the baseline derivation unit 106A may detect, from the pixel group detected by the waveform detection unit 105A, the coordinate value of the pixel located at the starting point of the P wave and the coordinate value of the pixel located at the starting point of the next P wave, and derive, as the baseline, an expression of a straight line connecting the detected coordinate values. For example, in the example of FIG. 6, the baseline derivation unit 106A detects the coordinate value of a starting point P1 of the first P wave included in the waveform W1 and the coordinate value of a starting point P2 of the second P wave, thereby deriving a straight line connecting these two points as the baseline L1.

For example, the baseline derivation unit 106A may detect a section in which numerical data is constant or substantially constant, calculate an average value μ of the y coordinate in the detected section, and derive a straight line represented by an expression of y=μ as a baseline. Since the value of the y-coordinate on the baseline of the waveform is constant or substantially constant, it is also possible to derive the baseline by such a method. For example, the baseline derivation unit 106A may calculate the values of the following mathematical expression for each of i=0, 1, . . . , and N using the minute bandwidth h and assuming the value of the y coordinate of the numerical data as yᵢ.

$${\sum }_{k = 1}^{i + h} \vert {y}_{k} - {y}_{k + 1} \vert \qquad [ Math 1 ]$$

Since the value of the above expression indicates the magnitude of variation of the numerical data near yᵢ, the baseline derivation unit 106A may detect a section in which the calculated value is small as a section in which the numerical data is constant or substantially constant. Then, the baseline derivation unit 106A may calculate the average value μ of the y coordinate in the detected section and derive a straight line represented by an expression of y=μ as a baseline.

### (Method of Specifying Scale)

The vertical direction (y-axis direction) in the waveform of the electrocardiogram indicates the strength of the potential (which can also be referred to as amplitude), and the potential of 1 mV is usually represented by 10 mm, but depending on the amplitude of the waveform, the potential of 1 mV may be represented by 5 mm, the potential of 1 mV may be represented by 2.5 mm, and the potential of 1 mV may be represented by 20 mm. Assuming that the scale in a case where the potential of 1 mV is represented by 10 mm is 1 time, the scale in a case where the potential of 1 mV is represented by 5 mm is ½ times, the scale in a case where the potential of 1 mV is represented by 2.5 mm is ¼ times, and the scale in a case where the potential of 1 mV is represented by 20 mm is 2 times. By considering such a scale, the coordinates of each pixel relevant to the portion of the waveform detected by the waveform detection unit 105A can be converted into appropriate numerical data.

To specify the scale, a reference object drawn such that a predetermined numerical value represented in a predetermined unit has a predetermined size is used. As described above, a waveform called a calibration wave is drawn on the electrocardiogram. The scale can be specified by using the calibration wave as the reference object.

For example, in the example of FIG. 6, the calibration wave W2 is drawn in association with the waveform W1, while a calibration wave W4 is drawn in association with a waveform W3. The height of the calibration wave W2 is H, whereas the height of the calibration wave W4 is H/2. All of these calibration waves indicate 1 mV. That is, the waveform W1 associated with the calibration wave W2 is displayed at a scale where 1 mV is H. On the other hand, the waveform W3 associated with the calibration wave W4 is displayed at a scale where 1 mV is H/2. That is, the waveform W3 is displayed at a scale ½ times the waveform W1 in the y-axis direction.

In a case where the calibration wave is used as the reference object, the scale specification unit 107A specifies the height of the calibration wave in the image data. For example, the scale specification unit 107A may specify a maximum value and a minimum value from among the values of the y coordinate of each pixel including the calibration wave, and may specify a value obtained by subtracting the specified minimum value from the specified maximum value as the height of the calibration wave. Then, the scale specification unit 107A specifies how many mV a waveform is drawn per unit height (one pixel) by dividing 1 mV by the specified height. For example, since the height of the calibration wave W2 drawn in association with the waveform W1 of FIG. 6 is H, the calibration wave W2 is specified to be drawn at a scale of “1/H” mV per unit height in the y-axis direction. On the other hand, the calibration wave W4 is specified to be drawn to a scale of “2/H” mV per unit height in the y-axis direction.

The scale in the y-axis direction can also be obtained from the distance between predetermined two points on the waveform. Here, the predetermined two points are points at which the potential difference between the two points is known. For example, in the example of FIG. 6, in a case where the peak value of the P wave is known, the scale in the y-axis direction can be specified using the height of the P wave and the known peak value.

### (Generation of Numerical Data Using Baseline and Scale)

The digitizing unit 101A calculates numerical data of the pixel by multiplying the distance in the y direction between the pixel including the waveform and the baseline derived by the baseline derivation unit 106A by the scale specified by the scale specification unit 107A. Since the baseline is usually parallel to the x axis, the distance in the y direction from the baseline can be calculated by a difference between the value of the y coordinate initially allocated to each pixel and the value of the y coordinate of the baseline. Therefore, in a case where the coordinates of the pixel including the waveform are (x1, y1), the value of the y-intercept of the baseline is y2, and the scale is s, numerical data relevant to the y-coordinate of this pixel is expressed as (y1−y2)×s. As described above, the process of generating numerical data can also be said to be a process of converting the coordinate values initially allocated to each pixel based on the baseline and the scale.

For example, the distance from the pixel located at a vertex P3 of the first peak in the waveform W1 of FIG. 6 to the baseline L1 is H_{A} (the difference between the value of the y coordinate of the vertex P3 and the value of the y-intercept of the baseline L1). As described above, the scale of the waveform W1 is specified as 1/H. Therefore, the numerical data relevant to the y coordinate of the pixel located at the vertex P3 is calculated as (1/H)×H_{A}=(H_{A}/H) mV. This value indicates the peak value of the first P wave in the waveform W1. On the other hand, the distance from the pixel located at a vertex P4 of the first peak in the waveform W3 to the baseline L3 is H_{B}. Therefore, the numerical data relevant to the y coordinate of the pixel located at the vertex P4 is calculated as (2/H)×H_{B}=(2H_{B}/H) mV. This value indicates the peak value of the first P wave in the waveform W3.

### (About Value of x Coordinate)

The digitizing unit 101A can similarly convert the value of the x coordinate of the pixel including the waveform into numerical data. The x-axis direction in the waveform of the electrocardiogram indicates time, and its unit is usually seconds. Therefore, the digitizing unit 101A converts the value of the x coordinate of the pixel including the waveform into numerical data in units of “seconds”. For conversion of the value of the x coordinate, a baseline perpendicular to the x axis and a scale in the x-axis direction are used.

For example, the baseline derivation unit 106A may derive, as the baseline, a straight line perpendicular to the x axis passing through the pixel having the smallest value of the x coordinate among the pixels included in the pixel group detected by the waveform detection unit 105A. A baseline L2 in the waveform W1 and a baseline L4 in the waveform W3 in FIG. 6 can be derived in this manner. The position of the baseline may be designated by the user. In this case, the baseline derivation unit 106A may derive, as a baseline, a straight line that passes through a position designated by the user and is perpendicular to the x axis.

The scale in the x-axis direction can be specified using a reference object drawn such that a predetermined numerical value represented in a predetermined unit has a predetermined size in the x-axis direction. For example, in a case where a grid drawn on the background of the waveform is detected as a reference object, the scale specification unit 107A can specify the scale in the x-axis direction from the detected width of the grid. For example, in a case where the width of one grid relevant to 0.04 seconds is w, the scale in the x-axis direction is specified as (0.04/w). The detection of the grid may be performed by the waveform detection unit 105A, or a detection unit for detecting the grid may be provided separately from the waveform detection unit 105A.

The scale in the x-axis direction can also be obtained from the distance between predetermined two points on the waveform. Here, the predetermined two points are points at which the time interval between the two points is known. For example, in the example of FIG. 6, the time from the point P1 to the point P2 is referred to as P-P time. If the P-P time is known, the scale in the x-axis direction can be specified using the distance between the point P1 and the point P2 in the x-axis direction and the P-P time.

As described above, the information processing device 1A may include a detection unit that detects, from the first image data, a reference object drawn such that a predetermined numerical value represented in a predetermined unit has a predetermined size. Then, based on the size of the detected reference object (more precisely, the width or height on the image data), the digitizing unit 101A may generate numerical data representing the time-series change of the biological signal in a predetermined unit. As a result, in addition to the effect obtained by the information processing device 1, it is possible to automatically generate numerical data having appropriate values for waveforms of various scales. As described above, the waveform detection unit 105A may be caused to detect the reference object, or a detection unit that detects the reference object may be provided separately from the waveform detection unit 105 A.

As described above, the information processing device 1A may include the baseline derivation unit 106A that derives a baseline passing through a plurality of points at which the biological signal has a predetermined reference value in the waveform. Then, the digitizing unit 101A may generate numerical data representing the time-series change of the biological signal in a predetermined unit based on the distance from the pixel detected by the waveform detection unit 105A to the baseline. As a result, in addition to the effect obtained by the information processing device 1, it is possible to automatically generate numerical data of an appropriate value based on the baseline.

The detection of the baseline and the specification of the scale are not necessarily performed automatically. In other words, it is not essential to provide the baseline derivation unit 106A, and it is not essential to provide the scale specification unit 107A. For example, either or both of the baseline and the scale may be designated by the user. In this case, the digitizing unit 101A may generate numerical data by applying a designated baseline and/or scale.

### (Modified Example of Method of Generating Numerical Data)

As described above, it is possible to generate numerical data by assigning a coordinate value to each pixel of the image data and then converting the coordinate value of each pixel of the pixel group relevant to the portion of the waveform. It is also possible to generate numerical data representing the time-series change of the biological signal in a predetermined unit relevant to the biological signal without performing such conversion.

For example, the digitizing unit 101A can also generate numerical data by setting, as a reference, a pixel whose value of a predetermined unit relevant to the biological signal is known among pixels included in the pixel group relevant to the waveform portion of the biological signal, and allocating a numerical value relevant to a positional relationship with the reference pixel to each pixel.

For example, it is assumed that coordinates of a left end point in the waveform W1 of FIG. 6 are (0 (seconds), 0 mV), one pixel in the x-axis direction is t (seconds), and one pixel in the y-direction is v (mV). These pieces of information may be input by the user or may be specified by analyzing image data.

In this case, the digitizing unit 101A may assign a numerical value relevant to the positional relationship with the reference point to another pixel included in the pixel group relevant to the waveform portion of the biological signal using the coordinates of the left end point in the waveform W1 as the reference point. Specifically, the digitizing unit 101A may allocate the numerical value of the x coordinate so as to increase by t every time one pixel is away from the reference point in the x-axis direction, and allocate the numerical value of the y coordinate so as to increase by v every time one pixel is away from the reference point in the y-axis direction. For example, the digitizing unit 101A may assign a numerical value (10t, 0) to a point 10 pixels away from the reference point in the x-axis direction. By allocating a numerical value to each point on the waveform W1 in this manner, the digitizing unit 101A can generate numerical data without performing conversion as described above.

### (Method of Generating Second Image Data)

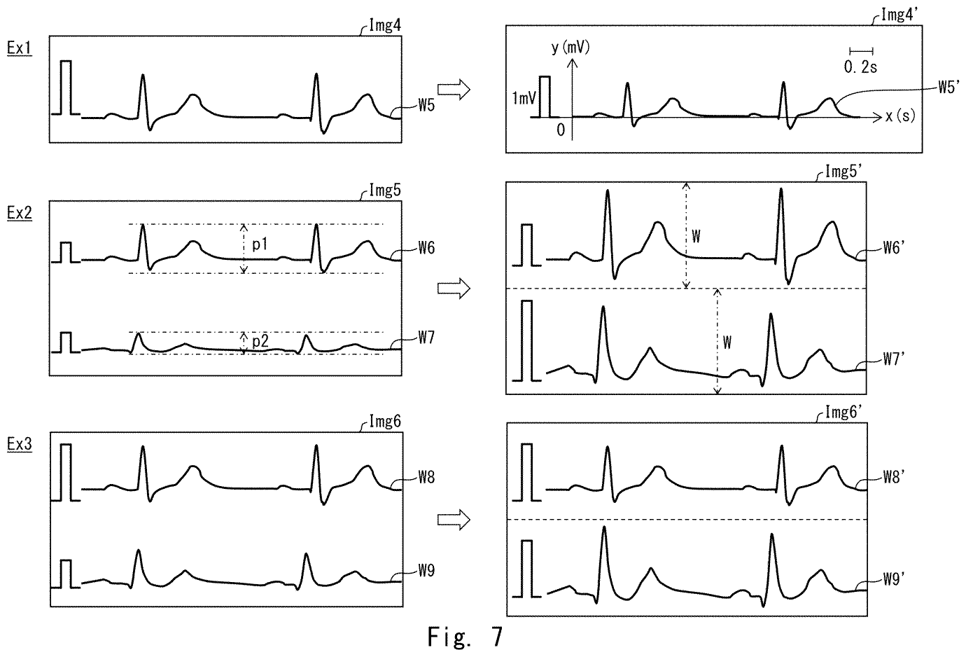

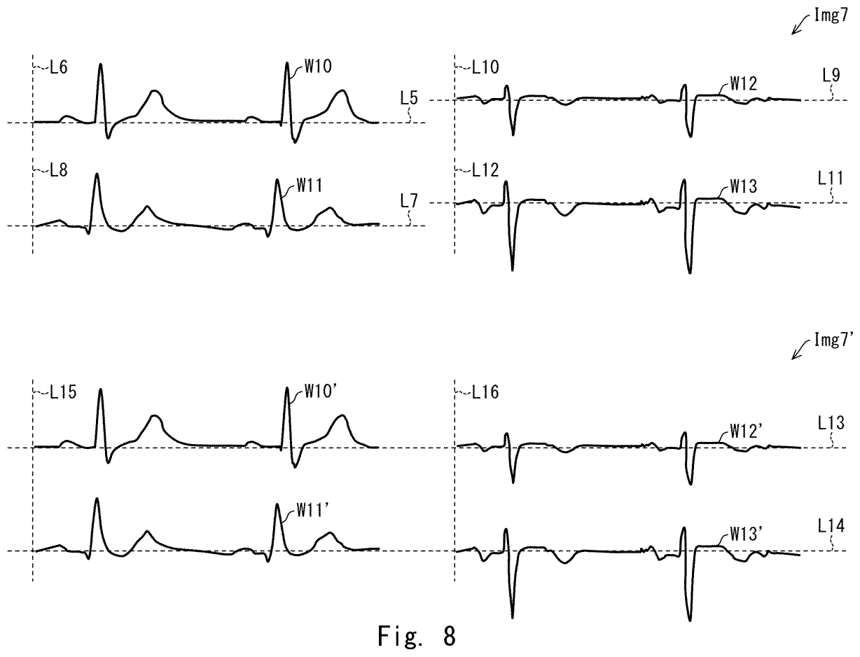

Since the numerical data generated by the digitizing unit 101A is data indicating the time-series change of the biological signal, the second image data indicating the waveform of the biological signal can be generated by using the numerical data. Various methods can be applied as a method of generating the second image data. Hereinafter, an example of a method of generating the second image data will be described with reference to FIG. 7.

FIG. 7 is a diagram illustrating an example of a method of generating second image data. More specifically, three examples Ex1 to Ex3 are illustrated in FIG. 7. In each of these examples, the target biological signal is the electrocardiogram, but the second image data can be generated similarly for biological signals other than the electrocardiogram.

Ex1 illustrated in FIG. 7 is an example in which numerical data generated by the digitizing unit 101A based on the first image data Img4 is plotted on a coordinate plane of a predetermined scale to generate second image data Img4′. In the second image data Img4′, a y axis in units of millivolts (mV) and an x axis in units of seconds(s) are set, and an intersection of these axes is indicated as an origin O. In the second image data Img4′, the numerical data generated by the digitizing unit 101A is plotted on the coordinate plane defined by the y axis and the x axis, whereby the waveform W5′ relevant to the waveform W5 is drawn.

In the second image data Img4′, a calibration wave is drawn and a scale bar indicating a scale in the x-axis direction is drawn. In this manner, the image generation unit 102A may draw the object indicating the scale in the second image together with the waveform. The scale of the second image data may be determined in advance or may be designated by the user. In the former case, there is an advantage that the second image data with the unified scale can be generated, and in the latter case, there is an advantage that the second image data with the scale desired by the user can be generated.

Ex2 illustrated in FIG. 7 is an example in which the second image data Img5′ is generated by drawing a waveform by applying a scale relevant to the size of the display area using the numerical data generated by the digitizing unit 101A based on the first image data Img5. As described above, the waveform of the electrocardiogram is basically represented by a potential of 1 mV as 10 mm, and is drawn at a scale of 2 times, ½ times, ¼ times, or the like according to the amplitude of the waveform. In the first image data Img5, it is assumed that the waveforms W6 and W7 are drawn at the same scale (½ times). The waveforms W6 and W7 are, for example, waveforms relevant to two of 12 types of leads.

Here, in the second image data Img5′, the widths of the display areas of the waveforms W6′ and W7′ relevant to the waveforms W6 and W7 are both W. Therefore, in the second image data Img5′, in a case where the scale is set to 1 time (1 mV is relevant to 10 mm), a waveform with a width (potential difference) of (W/10) at the maximum can be drawn. Therefore, if (W/10)≥p1, the waveform W6 in the first image data Img5 in which the potential difference from the maximum value to the minimum value is p1 can be drawn at a scale of 1. In a case where the scale is doubled (1 mV is relevant to 20 mm), a waveform having a width (potential difference) of (W/20) at the maximum can be drawn in the display area having a width of W, so that if (W/20)≥p1, the waveform W6 can be drawn even at a scale of 2.

As described above, based on the size of the display area and the width of the potential in the target waveform, the image generation unit 102A may obtain a scale that maximizes the waveform to be drawn, and draw the waveform by applying the scale. For example, if (W/10)≥p1 and (W/20)<p1 are satisfied for the waveform W6 of Ex2, the image generation unit 102A may draw the waveform W6′ as illustrated with the reduced scale set to 1 time. In a case where waveform W7 satisfies (W/20)≥p2 and (W/40)<p2, the image generation unit 102A may draw the waveform W7′ as illustrated with the reduced scale doubled. As a result, it is possible to generate the second image data Img5′ that easily recognizes the change in the details of the waveform while satisfying the constraint that the second image data Img5′ fits in the display area.

The scale does not necessarily need to be selected from a predetermined scale (for example, 2 times, 1 time, ½ times, ¼ times). For example, in a case where the width of the display area is W and the width of the potential in the target waveform is p, the image generation unit 102A may apply a scale in which the potential difference of p is represented by the width W. As a result, the waveform can be displayed to the full display area.

For example, in a case where it is desired to compare amplitudes between different types of leads, the scales of the plurality of waveforms may be made uniform. Therefore, as in Ex3 of FIG. 7, the image generation unit 102A may generate the second image data by applying the same scale to a plurality of waveforms included in the first image data and drawing the waveforms.

Specifically, in Ex3, the second image data Img6′ is generated based on the first image data Img6. In the first image data Img6, a waveform W8 is drawn at a scale of 1, and a waveform W9 is drawn at a scale of ½. On the other hand, in the second image data Img6′, the waveform W8′ relevant to the waveform W8 is drawn at a scale of 1 similarly to the first image data Img6, and the waveform W9′ relevant to the waveform W9 is also drawn at a scale of 1. As a result, in the second image data Img6′, it is easy to recognize that the waveform W9′ has a larger amplitude than the waveform W8′.

Any method may be used to determine the scale to be applied to the plurality of waveforms. For example, a scale to be applied may be determined in advance. For example, the image generation unit 102A may determine the scale according to the size of the display area of the waveform in the second image data. In this case, the image generation unit 102A may apply the largest scale within a range in which each waveform fits in the display area of each waveform.

As described above, the image generation unit 102A may generate the second image data by plotting the numerical data generated by digitizing unit 101A on a coordinate plane of a predetermined scale. As a result, in addition to the effect obtained by the information processing device 1, an effect that the second image data of a desired scale can be generated can be obtained. The above description “plotting numerical data on a coordinate plane of a predetermined scale” can also be rephrased as “drawing a waveform indicated by the numerical data on a coordinate system of a predetermined scale” or the like.

As described above, the image generation unit 102A may generate the second image data by plotting the numerical data generated by the digitizing unit 101A by applying the scale relevant to the size of the display area of the waveform in the second image data. As a result, in addition to the effect obtained by the information processing device 1, it is possible to generate second image data in which a waveform is drawn at an easily viewable scale according to the size of the display area of the waveform in the second image data. From the viewpoint of visibility, the image generation unit 102A may plot the numerical data generated by the digitizing unit 101A by applying the maximum scale that falls within the size of the display area of the waveform in the second image data.

Here, in a case where a plurality of waveforms are included in the first image data, in a case where the second image data is generated by applying different scales to the waveforms, the actual size difference between the waveforms is not reflected in the generated second image data. Therefore, in a case where the second image data generated by applying different scales to the plurality of waveforms is set as the training data, the training data generation unit 109A may also include the numerical data that is the source of the second image data in the training data. As a result, the characteristic of the size difference between the waveforms not reflected in the second image data can be compensated by the numerical data.

As described above, in a case where numerical data is generated from the first image data including a plurality of waveforms, the image generation unit 102A may generate the second image data by applying a common scale to the numerical data relevant to each waveform and plotting the numerical data. As a result, in addition to the effect obtained by the information processing device 1, it is possible to generate the second image data in which the magnitudes of the amplitudes of the plurality of waveforms can be easily compared.

In a case where the first image data includes a plurality of waveforms, the image generation unit 102A may draw waveforms in an arrangement order different from that of the first image data in the second image data. For example, in a case where the second image data is used for diagnosis, the image generation unit 102A may generate the second image data by plotting numerical data such that waveforms to be compared in the diagnosis are adjacent to each other. If there are two waveforms included in the first image data, the waveforms in the second image data are adjacent to each other, and thus such sorting is effective in a case where there are three or more waveforms included in the first image data. That is, in a case where numerical data is generated from first image data including three or more waveforms, the image generation unit 102A may generate second image data by plotting the numerical data such that waveforms to be compared are adjacent to each other. As a result, it is possible to generate second image data having high convenience in comparison and examination of waveforms. Since it is considered that a feature serving as a point of diagnosis appears in the second image data having high convenience in diagnosis by a person, such second image data is also suitable as training data of an inference model for diagnosis.

### (Unification of Positions of Baselines)

In a case where the first image data includes a plurality of waveforms, the positions of the baselines of the waveforms adjacent in the left-right direction may be shifted in the up-down direction. In this case, the image generation unit 102A may generate the second image data by plotting the numerical data such that the baselines of the waveforms adjacent in the left-right direction are aligned on a straight line. This will be described with reference to FIG. 8. FIG. 8 is a diagram illustrating another example of the method of generating the second image data.

The first image data Img7 illustrated in FIG. 8 is a part of the 12-lead electrocardiogram and includes waveforms W10 to W13. In the waveform W10, a baseline indicating a position where the potential is 0 is L5, and a baseline indicating a position where the time is 0 is L6. In the waveform W11, a baseline indicating a position where the potential is 0 is L7, and a baseline indicating a position where the time is 0 is L8. In the waveform W12, a baseline indicating a position where the potential is 0 is L9, and a baseline indicating a position where the time is 0 is L10. In the waveform W13, a baseline indicating a position where the potential is 0 is L11, and a baseline indicating a position where the time is 0 is L12. In the first image data Img7, the waveform W10 and the waveform W12 are adjacent in the left-right direction, and the baselines L5 and L9 thereof are shifted in the up-down direction (the y-axis direction or the amplitude direction). Similarly, the waveform W11 and the waveform W13 are adjacent to each other in the left-right direction, and the baselines L7 and L11 thereof are shifted in the up-down direction (the y-axis direction or the amplitude direction). The baseline in each waveform is derived by the baseline derivation unit 106A as described above.

By setting in advance a baseline serving as a reference for plotting numerical data relevant to each waveform on the coordinate plane of the second image data, it is possible to generate the second image data having no deviation as described above. Specifically, by causing the image generation unit 102A to plot numerical data with a preset baseline as a reference, it is possible to generate second image data in which the baselines of waveforms adjacent in the left-right direction are not shifted in the up-down direction.

For example, the second image data Img7′ illustrated in FIG. 8 is drawn with reference to the baselines L13 to L16. More specifically, in the second image data Img7′, numerical data relevant to the waveforms W10 and W12 is plotted such that the baseline L5 of the waveform W10 and the baseline L7 of the waveform W12 are both on the baseline L13. As a result, in the second image data Img7′, the baselines of the waveform W10′ and the waveform W12′ adjacent in the left-right direction are aligned on a straight line. The same applies to the waveform W11′ and the waveform W13′, and numerical data relevant to the waveforms is plotted such that the baseline L7 of the waveform W11 and the baseline L11 of the waveform W13 are both on the baseline L14. As a result, the baselines of the waveform W11′ and the waveform W13′ adjacent in the left-right direction are aligned on a straight line.

In the second image data Img7′, the baselines of the waveforms adjacent in the left-right direction are arranged on a straight line, so that the amplitudes of the waveforms can be easily compared. In a case where image data in which the positions of the baselines are displaced in various ways is included in the training data, there is a possibility that such a displacement is learned as a feature point of the image data and adversely affects the accuracy of the inference model. In this regard, if the second image data in which the baselines of adjacent waveforms are arranged on a straight line is used as the training data, such a risk can be avoided.

In FIG. 8, only four waveforms of the 12-lead electrocardiogram are illustrated, but the remaining eight waveforms can be similarly drawn. In the example of FIG. 8, regarding the waveforms W10 and W11 in the first image data Img7, the baselines L6 and L8 indicating the position where the time is 0 in these waveforms are arranged on a straight line. However, even in a case where these baselines are not arranged on a straight line, it is possible to generate second image data in which positions relevant to time 0 in waveforms adjacent in the up-down direction are aligned by plotting numerical data relevant to these waveforms such that the baselines L6 and L8 are on the baseline L15.

As described above, the information processing device 1A includes the baseline derivation unit 106A that derives a baseline passing through a plurality of points at which the biological signal has a predetermined reference value in the waveform. Then, in a case where numerical data is generated from first image data including a plurality of waveforms, the image generation unit 102A may generate second image data by plotting the numerical data such that baselines of adjacent waveforms are aligned on a straight line. As a result, in addition to the effect obtained by the information processing device 1, it is possible to obtain an effect of generating the second image data in which adjacent waveforms can be easily compared.

### (Display of Second Image Data)

As described above, the image generation unit 102A can generate the second image data in various display modes. Therefore, the display control unit 108A can display the second image data in various display modes generated by the image generation unit 102A. The displayed second image data can also be used for applications such as diagnosis. In this case, the image generation unit 102A may generate second image data indicating one or more waveforms designated by the user (diagnostician) among a plurality of waveforms (for example, 12 waveforms relevant to 12 types of leads). Then, the display control unit 108A may display the generated second image data. As a result, the waveform to be displayed can be narrowed down to a waveform that the user wants to check.

### (Flow of Processing)

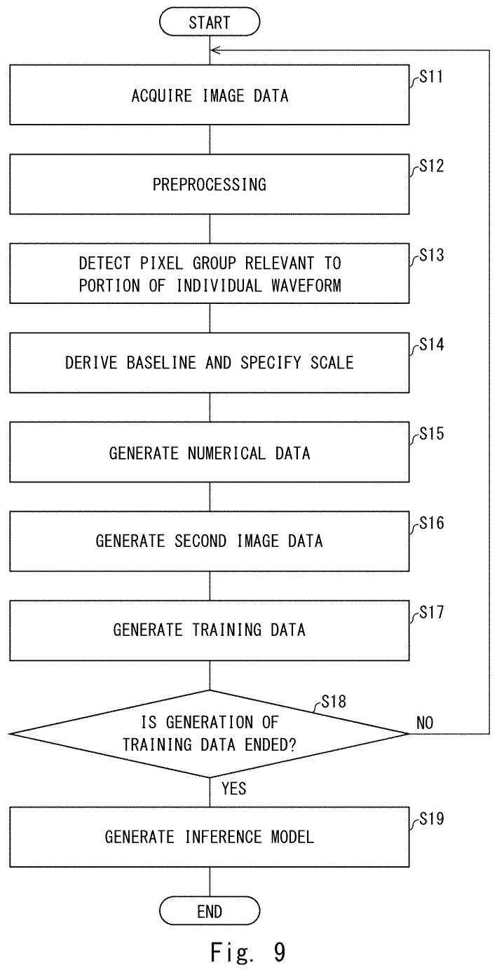

A flow of the processing executed by the information processing device 1A will be described with reference to FIG. 9. FIG. 9 is a flowchart illustrating an example of processing (inference model generation method) executed by the information processing device 1A. The flowchart of FIG. 9 includes each processing of the image generation method according to the present illustrative example embodiment, and also includes each processing of the training data generation method and the inference model generation method according to the present illustrative example embodiment.

In S11, the data acquisition unit 103A acquires image data indicating a waveform. The image data acquired here is before preprocessing by the preprocessing unit 104A is performed, and is relevant to the above-described “original image data”. For example, the data acquisition unit 103A may acquire original image data including a display area of an element other than a waveform, such as the original image data Img1 of FIG. 4. The data acquisition unit 103A may acquire image data subjected to preprocessing by the information processing device 1A or another device, and in this case, the processing of S12 is omitted.

In S12, the preprocessing unit 104A performs predetermined preprocessing on the image data (original image data) acquired in S11 to remove elements other than the waveform. The first image data is generated by the processing of S12. For example, in a case where the image data acquired in S11 is the original image data Img1 illustrated in FIG. 4, the preprocessing unit 104A may perform preprocessing of extracting the second area Ar2 from the original image data Img1 and binarizing the extracted area. As a result, the first image data such as the image data Img2 in FIG. 5 is generated. For example, the preprocessing unit 104A may perform masking processing on an area where characters, symbols, and the like are drawn in the image data Img2 to generate the first image data such as the image data Img3 of FIG. 5. The present illustrative example embodiment illustrates an example in which one preprocessing unit 104A performs a plurality of types of preprocessing, but a preprocessing unit may be provided for each type of preprocessing.

In S13, the waveform detection unit 105A assigns coordinate values to each pixel included in the first image data generated in S12, and then detects a pixel group relevant to the waveform portion of the biological signal from the first image data. Since coordinate values are allocated to each pixel, it can be said that a coordinate value group indicating the waveform of the biological signal is detected in S13.

In a case where the first image data generated in S12 includes a plurality of waveforms such as the image data Img2 and Img3 in FIG. 5, the waveform detection unit 105A detects each of pixel groups relevant to the plurality of waveforms in S13. In S13, the waveform detection unit 105A may detect a reference object (more precisely, a pixel group relevant to the reference object) drawn such that a predetermined numerical value represented in a predetermined unit has a predetermined size, such as the calibration waves W2 and W4 illustrated in FIG. 6.

In S14, the baseline derivation unit 106A derives a baseline serving as a reference for generating numerical data representing a time-series change of the biological signal in a predetermined unit relevant to the biological signal. For example, the baseline derivation unit 106A may detect a plurality of pixels at the position of the start point of the P wave from the pixels detected in S13, and derive a straight line connecting the pixels as a baseline (a baseline serving as a reference for generating numerical data in the y-axis direction). For example, the baseline derivation unit 106A may detect a pixel having the smallest x coordinate among the pixels detected in S13, and derive a straight line passing through the detected pixel and perpendicular to the x axis as a baseline (baseline serving as a reference for generating numerical data in the x-axis direction). In a case where a plurality of pixel groups have been detected in S13, the baseline derivation unit 106A derives a baseline for each pixel group (which can also be referred to as each waveform).

In S14, the scale specification unit 107A specifies the scale (both in the x-axis direction and the y-axis direction) in the first image data generated in S12. For example, in a case where a reference object is detected in S13, the scale specification unit 107A may specify the scale from the detected size of the reference object. As described above, the grid drawn on the background of the waveform may be used as the reference object. In this case, the grid may be detected from the original image data from which the grid has not been removed, and the scale may be specified from the size (width) of the detected grid. The specification of the scale and the derivation of the baseline may be independent steps.

In a case where a plurality of pixel groups have been detected in S13, the scale specification unit 107A specifies the scale for each pixel group (which can also be referred to as each waveform). However, for a pixel group having a common scale, the scale may be specified only for any one of the pixel groups. For example, in a case where the scale in the x-axis direction is common to a plurality of pixel groups, the scale in the x-axis direction may be specified only for one pixel group, and the same scale may be applied to the other pixel groups.

In S15 (digitizing processing), the digitizing unit 101A generates numerical data indicating the time-series change of the biological signal from the first image data indicating the waveform of the biological signal. More specifically, based on the detection result in S12, the baseline derived in S14, and the scale specified in S14, the digitizing unit 101A generates numerical data representing the time-series change of the biological signal in a predetermined unit relevant to the biological signal. For example, for each of the x coordinate and the y coordinate of the pixel included in the pixel group relevant to one waveform, the digitizing unit 101A calculates a distance between the coordinate and the baseline, and multiplies the calculated distance by the scale specified in S14. By performing this processing for each pixel included in the pixel group, numerical data representing the time-series change of the biological signal is generated in a predetermined unit relevant to the biological signal. In a case where a plurality of pixel groups have been detected in S13, the digitizing unit 101A generates numerical data for each pixel group (which can also be referred to as each waveform).

In S16 (image generation processing), the image generation unit 102A generates second image data using the numerical data generated in S15. As described above, the second image data indicates the same waveform of the biological signal as the first image data, but is image data different from the first image data. In the case of generating the second image data used as the training data as illustrated in FIG. 9, the image generation unit 102A may generate the second image data (for example, image data that does not include an element other than a waveform such as a calibration wave, a scale bar, or a grid line) in a predetermined format suitable for the training data.

In S17 (training data generation processing), the training data generation unit 109A generates training data for machine learning of the inference model using the second image data generated in S16. This inference model is a model for inferring the state of the subject based on image data indicating the waveform of the biological signal of the subject. In a case where the second image data includes a plurality of waveforms, the training data generation unit 109A may cut out a portion relevant to each waveform, and generate training data by associating a correct answer label with the cut out portion. In this case, the training data is generated according to the number of waveforms included in the second image data. The training data generation unit 109A may generate training data by associating a correct answer label with the second image data including a plurality of waveforms. In this case, one training data is generated for one second image data.

In S18, the training data generation unit 109A determines whether to end the generation of the training data. Conditions for ending generation of the training data may be appropriately determined. For example, the training data generation unit 109A may determine to end the generation of the training data (YES in S18) on the condition that the generation of a predetermined number of training data necessary for generating the inference model has been completed. If determined as YES in S18, the process proceeds to S19, and if determined as NO in S18, the process proceeds to S11. In S11 after the transition from S18, new first image data is acquired by the data acquisition unit 103A. The new first image data may have the same format or the like as that of the previously acquired first image data, or may have a different format or the like. Even if the new first image data is different in format or the like from the previously acquired first image data, the second image data having the same format or the like as the second image data generated from the previously acquired first image data is generated in S16.

In S19 (inference model generation processing), the learning unit 110A generates an inference model for inferring the state of the subject based on the image data indicating the waveform of the biological signal of the subject by machine learning using the training data generated in S18. Thereby, the processing of FIG. 9 is ended.

Inference using the above inference model can be performed by a flow similar to the above flow. In that case, in S11, the image data indicating the waveform of the biological signal of the subject is acquired as the first image data, the second image data generated in S16 is input to the inference model, and the inference result may be output. The second image data can be presented to the user by the same flow as described above. In this case, after S16, the display control unit 108A displays the second image data generated in S16. In this case, a step of receiving designation by the user of the display mode and the waveform to be displayed may be included before S16.

As described above, the training data generation method according to the present illustrative example embodiment includes the digitizing processing of generating numerical data indicating the time-series change of the biological signal from the first image data indicating the waveform of the biological signal, the image generation processing of generating the second image data indicating the waveform of the biological signal using the generated numerical data, and the training data generation processing of generating the training data for machine learning of the inference model for inferring the state of the subject based on the image data indicating the waveform of the biological signal of the subject using the generated second image data. As a result, it is possible to generate the training data including the second image data in which the format and the like are unified using the plurality of first image data in which the format and the like are not unified.

As described above, the inference model generation method according to the present illustrative example embodiment includes the digitizing processing of generating numerical data indicating a time-series change of the biological signal from first image data indicating a waveform of the biological signal, the image generation processing of generating second image data indicating a waveform of the biological signal using the generated numerical data, and the inference model generation processing of generating an inference model that infers a state of the subject based on image data indicating a waveform of the biological signal of the subject by machine learning using training data generated from the second image data. As a result, it is possible to obtain an effect that an inference model can be generated using a plurality of pieces of first image data of which formats and the like are not unified.

### MODIFIED EXAMPLES

An executing entity of each processing described in the above-described illustrative example embodiments is optional, and is not limited to the above-described examples. For example, a system having functions similar to those of the information processing devices 1 and 1A can be constructed by a plurality of devices capable of communicating with each other. The executing entity of each processing illustrated in the flowchart of FIG. 9 may be one device (may be rephrased as a processor) or a plurality of devices (may be similarly rephrased as processors).

### [Implementation Example by Software]

Some or all of the functions of the information processing devices 1 and 1A (hereinafter, also referred to as “each of the above devices”) may be implemented by hardware such as an integrated circuit (IC chip) or may be implemented by software.

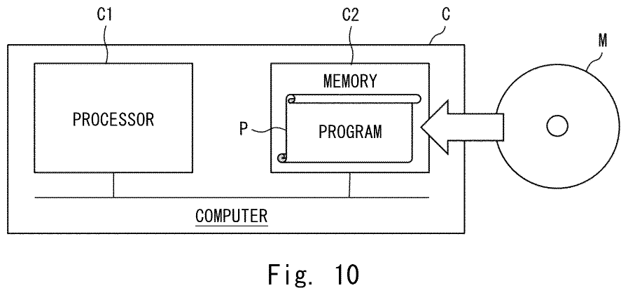

In the latter case, each of the above devices is implemented by, for example, a computer that executes commands of a program, that is software for implementing each function. An example of such a computer (which will be referred to as a computer C hereinafter) is illustrated in FIG. 10. FIG. 10 is a block diagram illustrating a hardware configuration of the computer C that functions as each of the above devices.

The computer C includes at least one processor C1 and at least one memory C2. A program (image generation program/training data generation program/inference model generation program) P for operating the computer C as each of the above devices is recorded in the memory C2. In the computer C, each function of each of the above devices is implemented by the processor C1 reading the program P from the memory C2 and executing the program P.

As the processor C1, for example, a central processing unit (CPU), a graphic processing unit (GPU), a digital signal processor (DSP), a micro processing unit (MPU), a floating point number processing unit (FPU), a physics processing unit (PPU), a tensor processing unit (TPU), a quantum processor, a microcontroller, or a combination of these can be used. As the memory C2, for example, a flash memory, a hard disk drive (HDD), a solid state drive (SSD), or a combination of these can be used.

The computer C may further include a random access memory (RAM) for loading the program P at the time of execution and temporarily storing various types of data. The computer C may further include a communication interface for transmitting and receiving data to and from another device. The computer C may further include an input/output interface for connecting input/output devices such as a keyboard, a mouse, a display, and a printer.

The program P can be recorded in a non-transitory tangible recording medium M readable by the computer C. As such a recording medium M, for example, a tape, a disk, a card, a semiconductor memory, a programmable logic circuit, or the like can be used. The computer C can acquire the program P via such a recording medium M. The program P can be transmitted via a transmission medium. As such a transmission medium, for example, a communication network, a broadcast wave, or the like can be used. The computer C can also acquire the program P via such a transmission medium.

Each of the above functions of each of the above devices may be achieved by a single processor provided in a single computer, may be achieved in cooperation with a plurality of processors provided in a single computer, or may be achieved in cooperation with a plurality of processors provided in a plurality of computers. The program for causing each of the above devices to achieve each of the above functions may be stored in a single memory provided in a single computer, may be stored in a distributed manner in a plurality of memories provided in a single computer, or may be stored in a distributed manner in a plurality of memories provided in a plurality of computers.

### SUPPLEMENTARY INFORMATION

The present disclosure includes the techniques described in the following supplementary notes. However, the present invention is not limited to the technologies described in the following Supplementary Notes, and various modifications can be made within the scope described in the claims.

### Supplementary Note A1

An information processing device including: a digitizing means for generating numerical data indicating a time-series change of a biological signal from first image data indicating a waveform of the biological signal; and an image generation means for generating second image data indicating a waveform of the biological signal by using the numerical data.

### Supplementary Note A2

The information processing device according to Supplementary Note A1, in which the image generation means generates the second image data by plotting the numerical data on a coordinate plane of a predetermined scale.

### Supplementary Note A3

The information processing device according to Supplementary Note A1 or A2, including a baseline derivation means for deriving a baseline passing through a plurality of points at which the biological signal has a predetermined reference value in the waveform, in which in a case where the numerical data is generated from the first image data including a plurality of waveforms, the image generation means generates the second image data by plotting the numerical data such that baselines of adjacent waveforms are aligned on a straight line.

### Supplementary Note A4

The information processing device according to any one of Supplementary Notes A1 to A3, in which the image generation means generates the second image data by plotting the numerical data by applying a scale relevant to a size of a display area of a waveform in the second image data.

### Supplementary Note A5

The information processing device according to any one of Supplementary Notes A1 to A3, in which in a case where the numerical data is generated from the first image data including a plurality of waveforms, the image generation means generates the second image data by plotting numerical data relevant to the waveforms by applying a common scale.

### Supplementary Note A6

The information processing device according to any one of Supplementary Notes A1 to A5, in which in a case where the numerical data is generated from the first image data including three or more waveforms, the image generation means generates the second image data by plotting the numerical data in such a way that waveforms to be compared are adjacent to each other.

### Supplementary Note B1

An image generation method for causing at least one processor to execute: digitizing processing of generating numerical data indicating a time-series change of a biological signal from first image data indicating a waveform of the biological signal; and image generation processing of generating second image data indicating a waveform of the biological signal by using the numerical data.

### Supplementary Note B2

The image generation method according to Supplementary Note B1, in which in the image generation processing, the at least one processor generates the second image data by plotting the numerical data on a coordinate plane of a predetermined scale.

### Supplementary Note B3

The image generation method according to Supplementary Note B1 or B2, in which the at least one processor includes baseline derivation processing of deriving a baseline passing through a plurality of points at which the biological signal has a predetermined reference value in the waveform, and in the image generation processing, in a case where the numerical data is generated from the first image data including a plurality of waveforms, the at least one processor generates the second image data by plotting the numerical data such that baselines of adjacent waveforms are aligned on a straight line.

### Supplementary Note B4

The image generation method according to any one of Supplementary Notes B1 to B3, in which in the image generation processing, the at least one processor generates the second image data by plotting the numerical data by applying a scale relevant to a size of a display area of a waveform in the second image data.

### Supplementary Note B5

The image generation method according to any one of Supplementary Notes B1 to B3, in which in the image generation processing, in a case where the numerical data is generated from the first image data including a plurality of waveforms, the at least one processor generates the second image data by plotting numerical data relevant to the waveforms by applying a common scale.

### Supplementary Note B6

The image generation method according to any one of Supplementary Notes B1 to B5, in which in the image generation processing, in a case where the numerical data is generated from the first image data including three or more waveforms, the at least one processor generates the second image data by plotting the numerical data in such a way that waveforms to be compared are adjacent to each other.

### Supplementary Note C1

An image generation program for causing a computer to function as: a digitizing means for generating numerical data indicating a time-series change of a biological signal from first image data indicating a waveform of the biological signal; and an image generation means for generating second image data indicating a waveform of the biological signal by using the numerical data.

### Supplementary Note C2

The image generation program according to Supplementary Note C1, in which the image generation means generates the second image data by plotting the numerical data on a coordinate plane of a predetermined scale.

### Supplementary Note C3

The image generation program according to Supplementary Note C1 or C2, the program causing the computer to function as a baseline derivation means for deriving a baseline passing through a plurality of points at which the biological signal has a predetermined reference value in the waveform, in which in a case where the numerical data is generated from the first image data including a plurality of waveforms, the image generation means generates the second image data by plotting the numerical data such that baselines of adjacent waveforms are aligned on a straight line.

### Supplementary Note C4

The image generation program according to any one of Supplementary Notes C1 to C3, in which the image generation means generates the second image data by plotting the numerical data by applying a scale relevant to a size of a display area of a waveform in the second image data.

### Supplementary Note C5

The image generation program according to any one of Supplementary Notes C1 to C3, in which in a case where the numerical data is generated from the first image data including a plurality of waveforms, the image generation means generates the second image data by plotting numerical data relevant to the waveforms by applying a common scale.

### Supplementary Note C6

The image generation program according to any one of Supplementary Notes C1 to C5, in which in a case where the numerical data is generated from the first image data including three or more waveforms, the image generation means generates the second image data by plotting the numerical data in such a way that waveforms to be compared are adjacent to each other.

### Supplementary Note D1

An information processing device including at least one processor, in which the at least one processor executes: digitizing processing of generating numerical data indicating a time-series change of a biological signal from first image data indicating a waveform of the biological signal; and image generation processing of generating second image data indicating a waveform of the biological signal by using the numerical data.

The information processing device may further include a memory. The memory may store a program for causing the at least one processor to execute each processing.

### Supplementary Note D2

The information processing device according to Supplementary Note D1, in which in the image generation processing, the at least one processor generates the second image data by plotting the numerical data on a coordinate plane of a predetermined scale.

### Supplementary Note D3

The information processing device according to Supplementary Note D1 or D2, in which
baseline derivation processing is executed to derive a baseline passing through a plurality of points at which the biological signal has a predetermined reference value in the waveform, and in the image generation processing, in a case where the numerical data is generated from the first image data including a plurality of waveforms, the at least one processor generates the second image data by plotting the numerical data such that baselines of adjacent waveforms are aligned on a straight line.

### Supplementary Note D4

The information processing device according to any one of Supplementary Notes D1 to D3, in which in the image generation processing, the at least one processor generates the second image data by plotting the numerical data by applying a scale relevant to a size of a display area of a waveform in the second image data.

### Supplementary Note D5

The information processing device according to any one of Supplementary Notes D1 to D3, in which in the image generation processing, in a case where the numerical data is generated from the first image data including a plurality of waveforms, the at least one processor generates the second image data by plotting numerical data relevant to the waveforms by applying a common scale.

### Supplementary Note D6

The information processing device according to any one of Supplementary Notes D1 to D5, in which in the image generation processing, in a case where the numerical data is generated from the first image data including three or more waveforms, the at least one processor generates the second image data by plotting the numerical data in such a way that waveforms to be compared are adjacent to each other.

### Supplementary Note E

A non-transitory recording medium having recorded therein an image generation program for causing a computer to execute: a digitizing processing of generating numerical data indicating a time-series change of a biological signal from first image data indicating a waveform of the biological signal; and an image generation processing of generating second image data indicating a waveform of the biological signal by using the numerical data.

1. An information processing device comprising:
at least one memory storing instructions and
at least one processor configured to execute the instructions to:
receive first image data, the first image data comprising a graphical representation of a waveform of a biological signal and a graphical reference object indicating a physical scale of the biological signal;
detect, from the first image data, a pixel group corresponding to the graphical representation of the waveform;
determine, based on a dimension of the graphical reference object within the first image data, a conversion scale for converting pixel coordinates to physical units of the biological signal;
generate, by applying the determined conversion scale to coordinates of pixels in the detected pixel group, numerical data indicating a time-series change of the biological signal in the physical units; and
generate, from the numerical data, second image data by drawing a waveform of the biological signal in a predetermined coordinate system defined by the physical units, regardless of a pixel arrangement of the first image data.

2. The information processing device according to claim 1, wherein the at least one processor is further configured to execute the instructions to generate the second image data by plotting the numerical data on a coordinate plane of a predetermined scale in the physical units.

3. The information processing device according to claim 1, wherein the at least one processor is further configured to execute the instructions to:
derive a baseline passing through a plurality of points at which the biological signal has a predetermined reference value in the waveform, based on the detected pixel group,
generate, in a case where the numerical data is generated from the first image data including a plurality of waveforms, the second image data by plotting the numerical data such that baselines of adjacent waveforms are aligned on a straight line.

4. The information processing device according to claim 1, wherein the at least one processor is further configured to execute the instructions to generate the second image data by plotting the numerical data by applying a scale for mapping the physical units to pixels in accordance with a size of a display area of a waveform in the second image data.

5. The information processing device according to claim 1, wherein the at least one processor is further configured to execute the instructions to generate, in a case where the numerical data is generated from the first image data including a plurality of waveforms, the second image data by plotting numerical data relevant to the waveforms by applying a common scale in the physical units.

6. The information processing device according to claim 1, wherein the at least one processor is further configured to execute the instructions to generate, in a case where the numerical data is generated from the first image data including three or more waveforms, the second image data by plotting the numerical data in such a way that waveforms to be compared are adjacent to each other.

7. An image generation method for causing at least one processor to execute:
receiving first image data, the first image data comprising a graphical representation of a waveform of a biological signal and a graphical reference object indicating a physical scale of the biological signal;
detecting, from the first image data, a pixel group corresponding to the graphical representation of the waveform;
determining, based on a dimension of the graphical reference object within the first image data, a conversion scale for converting pixel coordinates to physical units of the biological signal;
generating, by applying the determined conversion scale to coordinates of pixels in the detected pixel group, numerical data indicating a time-series change of the biological signal in the physical units; and
generating, from the numerical data, second image data by drawing a waveform of the biological signal in a predetermined coordinate system defined by the physical units, regardless of a pixel arrangement of the first image data.

8. A non-transitory computer-readable medium storing an image generation program for causing a computer to execute:
receive first image data, the first image data comprising a graphical representation of a waveform of a biological signal and a graphical reference object indicating a physical scale of the biological signal;
detect, from the first image data, a pixel group corresponding to the graphical representation of the waveform;
determine, based on a dimension of the graphical reference object within the first image data, a conversion scale for converting pixel coordinates to physical units of the biological signal;
generate, by applying the determined conversion scale to coordinates of pixels in the detected pixel group, numerical data indicating a time-series change of the biological signal in the physical units; and
generate, from the numerical data, second image data by drawing a waveform of the biological signal in a predetermined coordinate system defined by the physical units, regardless of a pixel arrangement of the first image data.
